(19) United States
(12) Patent Application Publication
(10) Pub. No.: US 2013/0303602 A1
(43) Pub. Date: Nov. 14, 2013

(54) NOVEL PTEROCARPAN COMPOUND OR PHARMACEUTICALLY ACCEPTABLE SALT THEREOF AND PHARMACEUTICAL COMPOSITION FOR PREVENTION OR TREATMENT OF METABOLIC DISEASE OR COMPLICATION THEREOF, OR FOR ANTIOXIDANT CONTAINING THE SAME AS AN ACTIVE INGREDIENT

(71) Applicants: Tae-Sook JEONG, Daejeon (KR); Ho-Yong PARK, Daejeon (KR); Ki Hun PARK, Jinju-si (KR)

(72) Inventors: Tae-Sook JEONG, Daejeon (KR); Ho-Yong PARK, Daejeon (KR); Ki Hun PARK, Jinju-si (KR)

(73) Assignee: KOREA RESEARCH INSTITUTE OF BIOSCIENCE AND TECHNOLOGY, Daejeon (KR)

(21) Appl. No.: 13/895,760

(22) Filed: May 16, 2013

Related U.S. Application Data
(63) Continuation-in-part of application No. PCT/KR2011/002476, filed on Apr. 8, 2011.

(30) Foreign Application Priority Data
Nov. 17, 2010 (KR) 10-2010-0114673

(51) Int. Cl. C07D 493/04

(52) U.S. Cl. CPC C07D 493/04
USPC 514/453; 549/383

(57) ABSTRACT

The present invention relates to novel pterocarpan compound or pharmaceutically acceptable salt thereof and a composition for the prevention or treatment of metabolic disease or complications thereof comprising the same as an active ingredient. The novel pterocarpan compound of the present invention isolated from soybean leaves inhibits α-glucosidase activity and hACAT activity, and suppresses LDL-oxidation efficiently. Therefore, the compound of the present invention not only can be effectively used for the prevention or treatment of metabolic disease or complications thereof but also can be effectively used as an anti-oxidative composition owing to its excellent anti-oxidative activity.

### BACKGROUND OF THE INVENTION

1. Field of the Invention

The present invention relates to novel pterocarpan compound or a pharmaceutically acceptable salt thereof and a pharmaceutical composition for prevention or treatment of metabolic disease or complications thereof, or for anti-oxidation containing the same as an active ingredient.

2. Description of the Related Art

Along with the rapid changes and advancement of modern society and richness in nutrients, human metabolic environment has been significantly changed from the original condition of primitive times. The prevalence rate of metabolic disease that is called adult disease or modern disease characterized by co-occurrence of two or more diseases such as obesity, type 2 diabetes, hypertension, hypertriglyceridemia, hypercholesterolemia, and arteriosclerosis reached 32.3% (male: 32.9%, female: 31.8%) in 2005, according to the National Health and Nutrition Survey, and heart disease and stroke caused by such metabolic disease are on the rise, taking the second and the third place in cause of death in Korea.

When human body loses its balance in metabolism, metabolic waste and toxin are not released but accumulated. Metabolic disease is developed when human body loses its functions of each organ because of the metabolic waste accumulation, which is progressed as metabolic syndrome, also known as insulin resistance syndrome. Metabolic syndrome does damage to coronary artery, leading to heart disease or stroke, decreases salt elimination function in the kidney to cause hypertension, increases neutral fat that can cause cardiovascular disease, increases risk of blood coagulation, and suppresses insulin synthesis with causing type 2 diabetes, resulting in the damage in the eye, the kidney, and the nerve.

Diabetes is the disease characterized by high blood sugar so that glucose is discharged from the body as urine. This disease is developed by abnormal metabolism of insulin in β-cells of Langerhans islets in the pancreas or abnormal physiological activity. Diabetes is grouped as insulin dependent diabetes and insulin non-dependent diabetes according to the insulin secretion and the function of the same. In the case of insulin dependent diabetes (type 1 diabetes), pancreatic β-cells are destroyed so that insulin is not secreted, indicating severe insulin shortage. Therefore, insulin has to be provided regularly from outside to prevent ketosis and death. This type of diabetes is developed before an adolescent period, because of which it is also called childhood diabetes. But, at least 90% of diabetes patients suffer from the insulin non-dependent diabetes (type 2 diabetes). In the case of type 2 diabetes, pancreatic β-cells can still function to secret insulin but insulin itself cannot function normally. Diabetes makes high blood sugar condition in human body that causes osmotic stress severely enough to cause complications such as cataract and renal disease (Campbell, R. K. and Steil, C. F. 1988. Diabetes, clinical pharmacy and therapeutics. William & Wilkins. 4, p. 176). Once diabetes is developed, imbalance in such hormone as insulin, glucagon, and glucocorticoid causes physiological abnormality or abnormal metabolism including carbohydrate, protein, lipid, and electrolyte metabolisms, with developing typical diabetes symptoms like high blood sugar and glycosuria, etc. The representative complications of diabetes are vascular dysfunction, neurological disorder, and infection. Alpha-glucosidase is the enzyme involved in the last phase of carbohydrate digestion. The alpha-glucosidase inhibitors suppress carbohydrate absorption, so that they can inhibit instant blood sugar increase after a meal. Therefore, the α-glucosidase inhibitors have been used for regulating diabetes and obesity (Int. J. Obes., 11(Supple 2): 28, 1987). The most common α-glucosidase inhibitor is acarbose that has been used as an oral antidiabetic agent (Digestion, 23: 232-238, 1982). However, it can induce side effects such as hypoglycemia shock, gas generation, and intestinal dysfunction. Thus, it is requested to develop a safer hypoglycemic agent with less side effects.

Obesity, one of chronic diseases of modern people, is regarded to be caused by changes in diet habit and life style according to the industrialization and great increase of income level, which becomes now the most serious health problem world-wide. Even, obesity was designated as a disease by WHO in 1996. Obesity has been believed to associate directly or indirectly with the onset of adult diseases such as diabetes, hypertension, hyperlipidemia, and heart disease and various cancers.

One of many causes of metabolic syndrome is cholesterol. Acyl-CoA: cholesterol acyltransferase (ACAT) plays a role in accumulation of cholesterol in the form of ester in cells, which is exemplified by hACAT-1 and hACAT-2. hACAT-1 (50 kDa) functions mainly in the liver, adrenal gland, macrophages, and kidney of adults, while hACAT-2 (46 kDa) functions in the small intestine (Curr. Opin. Lipidol., 12: 121-127, 2001). A variety of new drugs are under development for the treatment of hypercholesterolemia, cholesterol stone, and arteriosclerosis by using the ACAT inhibitor based on its mechanism of suppressing cholesteryl ester accumulation in vascular wall (Nature Med., 6: 1341-1347, 2000).

Arteriosclerosis is caused easily by the increase of low-density lipoprotein (LDL) in cerebral artery or coronary artery. Arteriosclerosis might be progressed to cardiovascular disease such as heart disease and cerebrovascular disease. Plaque formation in the vascular endothelial wall and vascular rupture are major reasons of myocardial infarction. The early development of arteriosclerosis is suggested to be caused by the chronic inflammation process by the damage of vascular endothelial wall, indicating that it is rather explained by response-to-injury hypothesis, a defense mechanism, by than an injury mechanism (Circ. Res., 2001, 89, 298-304). That is, vascular endothelial cells cannot maintain normal homeostasis and fall into malfunction state because of genetic mutation, peroxide, hypertension, diabetes, increase of plasma homocysteine and/or microorganism infection.

More precisely, because of the causes above, LDL is converted into highly modified-LDL (HM-LDL) via oxidation, sugar-binding, integration, and glycoprotein-binding. HM-LDL stimulates vascular endothelial cells and smooth muscles and further causes injury therein. Accordingly, the expression of vascular cell adhesion molecule-1 (VCAM-1) and the release of inflammatory mediators are accelerated, resulting in the inflow and accumulation of LDL in endothelial cells. The accumulated LDL and oxidized HM-LDL induce the inflow and activation of immune cells such as macrophages and T-lymphocytes. At last, inflammation occurs in the lesion by the repeat of the above. Then, a series of actions is repeated as follows; The lesion is killed by the hydrolases, inflammatory mediators, and growth factors discharged from the macrophages or lymphocytes brought into the lesion and then mononuclear cells and smooth muscles are migrated and differentiated around the dead lesion and at the same time fibrous tissues are formed around the lesion. By the repeated procedure, lesional tissue is developed into more complicated fibrous lesion having HM-LDL as a core in the center and fibroid materials covering the dead tissues around. Thrombus is generated from the developed lesional tissue and artery becomes hardened, leading to cardiovascular disease including blood flow dysfunction.

LDL oxidation seems to be the most critical reason of early development of arteriosclerosis including atherosclerosis (Circulation, 1995, 91: 2488-2496; Arterioscler. Thromb. Vasc. Biol., 1997, 17: 3338-3346). Endogenous or exogenous oxidative stress converts blood LDL into oxidized-LDL, which migrates into intima through adhesion molecules. Monocytes eat the migrated oxidized-LDL to form foam cells, leading to the generation of fatty streak which is the early lesion of arteriosclerosis. The early lesion of arteriosclerosis is characterized by the expressions of VCAM-1, intracellular adhesion molecule-1 (ICAM-1), and monocyte chemoattractant protein-1 (MCP-1), which are induced by nuclear factor-κB (NF-κB) activated by various factors, particularly by the transcription factors such as active oxygen or cytokine, etc. The activated NF-κB binds to specific promoter gene along with Interlukin-1 (IL-1), VCAM-1, ICAM-1, and other factors involved in the progress of arteriosclerosis to regulate the expressions of various inflammatory factors. Antioxidants and free radical scavengers are known to inhibit NF-κB activation. So, studies have been actively undergoing based on that when an antioxidant is taken properly, in vivo LDL oxidation can be inhibited and the expressions of adhesion molecules are also inhibited, leading to the decrease of NF-κB activity, by which arteriosclerosis is expected to be suppressed (Korean Patent Publication No. 2004-0079206).

The study to eliminate the reason of LDL peroxide generation in hyperlipidemia, coronary artery disease, arteriosclerosis, and myocardial infarction patients has also been actively undergoing (Curr. Atheroscler. Res., 2000, 2: 363-372). The recent hyperlipidemia treating agents, Probucol and N,N′-diphenylenediamine, and the phenol synthetic antioxidants, BHA (butylated hydroxyanisol) and BHT (butylated hydroxytoluene) reduce LDL-cholesterol and decrease lesion formation by lowering oxidation level, but even if they have excellent anti-oxidative activity, these hyperlipidemia treating agents are limited in use because of their side effects.

To prevent such disease, attempts have been made to reduce plasma LDL by inhibiting cholesterol absorption and biosynthesis (Principles in Biochemistry, lipid biosynthesis, 770-817, 3rd Edition, 2000 Worth Publishers, New York; Steinberg, N. Engl. J. Med., 1989, 320: 915-924). The interest in the co-treatment of LDL antioxidants with lipid-lowering drugs to the patients with hyperlipidemia, coronary artery disease, arteriosclerosis, and myocardial infarction has also been growing.

The known pharmaceutical compositions for the prevention and treatment of metabolic disease are Alpinia katsumadai extracts (Korean Patent Publication No. 2010-115423), Scutellaria radix extracts and Platycodi radix extracts (Korean Patent Publication No. 2010-043537), benzazole derivatives or pharmaceutically acceptable salts thereof (Korean Patent No. 811100). However, it is still requested to develop a novel therapeutic agent effective in treating metabolic disease.

Korean Patent No. 1011454 describes the pharmaceutical compositions for prevention and treatment of influenza viral diseases containing pterocarpan compounds—Pterocarpin, Maackiain, Trifolrhizin—isolated from Sophora flavescens extracts as an active ingredient. However, the reports concerning the effect of pterocarpan compound on metabolic disease and complications thereof have not been reported, yet.

The present inventors screened therapeutic agents for metabolic disease or complications thereof from natural substances, in the middle of which the inventors confirmed that the pterocarpan compounds obtained from soybean leaf extracts had the inhibitory activities of α-glucosidase, hACAT-1 and hACAT-2, and LDL-oxidation. At last, the present inventors completed this invention by confirming that the pterocarpan compounds could be effectively used not only for the prevention or treatment of metabolic disease or complications thereof but also as an anti-oxidative composition.

### SUMMARY OF THE INVENTION

It is an object of the present invention to provide novel pterocarpan compound or pharmaceutically acceptable salt thereof.

It is another object of the present invention to provide a pharmaceutical composition for the prevention or treatment of metabolic disease or complications thereof containing the said pterocarpan compound or the pharmaceutically acceptable salt thereof as an active ingredient.

It is further an object of the present invention to provide a treatment method for metabolic disease or complications thereof using the said pterocarpan compound or the pharmaceutically acceptable salt thereof.

It is also an object of the present invention to provide a health functional food composition for the prevention or improvement of metabolic disease or complications thereof containing the said pterocarpan compound or the pharmaceutically acceptable salt thereof as an active ingredient.

It is also an object of the present invention to provide an anti-oxidative pharmaceutical composition, a health functional food composition, a cosmetic composition, or a feed additive, containing the said pterocarpan compound or the pharmaceutically acceptable salt thereof as an active ingredient.

To achieve the above-mentioned objects, the present invention provides the novel pterocarpan compound represented by the following formula 1 or a pharmaceutically acceptable salt thereof:

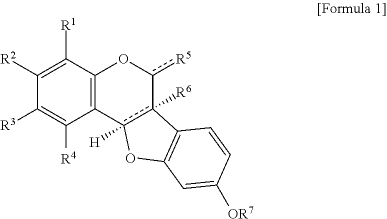

(In the Formula 1,

[symbol] , R¹, R², R³, R⁴, R⁵, R⁶ and R⁷ are as defined in this description).

The present invention also provides a pharmaceutical composition for the prevention or treatment of metabolic disease or complications thereof containing the pterocarpan compound represented by formula 1 or the pharmaceutically acceptable salt thereof as an active ingredient.

The present invention further provides a treatment method for metabolic disease or complications thereof containing the step of administering a therapeutically effective dose of the pterocarpan compound represented by formula 1 or the pharmaceutically acceptable salt thereof to a patient in need of treatment.

The present invention also provides a health functional food composition for the prevention or improvement of metabolic disease or complications thereof containing the pterocarpan compound represented by formula 1 or the pharmaceutically acceptable salt thereof as an active ingredient.

In addition, the present invention provides an anti-oxidative pharmaceutical composition, a health functional food composition, a cosmetic composition, or a feed additive, containing the pterocarpan compound represented by formula 1 or the pharmaceutically acceptable salt thereof as an active ingredient.

The novel pterocarpan compound of the present invention isolated from soybean leaf extracts inhibits the activities of α-glucosidase and hACAT-1 and hACAT-2, and suppresses LDL-oxidation efficiently, so that it can be effectively used not only for the prevention or treatment of metabolic disease or complications thereof but also as an anti-oxidative composition owing to its excellent anti-oxidative activity.

### BRIEF DESCRIPTION OF THE DRAWINGS

FIG. 1 is a flow chart illustrating the preparation method of the compound of the present invention.

### DETAILED DESCRIPTION OF THE INVENTION

Hereinafter, the present invention is described in detail.

The present invention provides the novel pterocarpan compound represented by the following formula 1 or a pharmaceutically acceptable salt thereof:

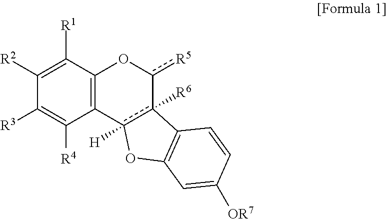

In the Formula 1,

[symbol] is single bond or double bond;

R¹ is hydrogen or isobutenyl group;

R² is hydroxyl group, and R³ is hydrogen or isobutenyl group, or R² and R³ can form 5-7 membered heteroaryl ring with carbon atoms conjugated with them in addition to 1 heteroatom selected from the group consisting of N, O, and S. At this time, the heteroaryl ring is non-replaceable or can be replaced with isopropenyl group;

R⁴ is hydrogen or C₁-C₄ straight or branched alkoxy group;

R⁵ is hydrogen or ═O;

R⁶ is hydrogen or hydroxyl group; and

R⁷ is hydrogen or C₁-C₄ straight or branched alkyl group.

More preferably, [symbol] is single bond or double bond;

R¹ is hydrogen or isobutenyl group;

R² is hydroxyl group, and R³ is hydrogen or isobutenyl group, or R² and R³ can form furan ring with carbon atoms conjugated with them, and at this time the furan ring is non-replaceable or can be replaced with isopropenyl group;

R⁴ is hydrogen or methoxy group;

R⁵ is hydrogen or ═O;

R⁶ is hydrogen or hydroxyl group; and

R⁷ is hydrogen or methyl group.

Most preferably, the pterocarpan compound represented by formula 1 is as follows:

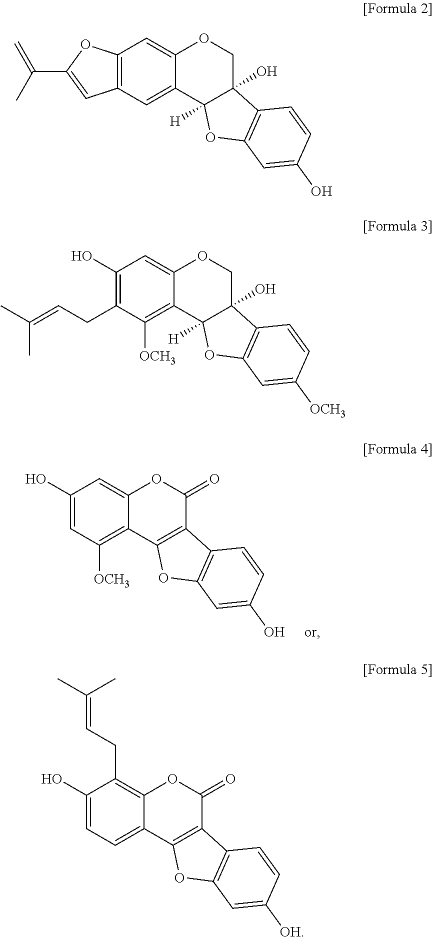 [SMILES: C=C(C)c1cc2cc3c(cc2o1)OC[C@@]1(O)c2ccc(O)cc2O[C@@H]31 ; COc1ccc2c(c1)O[C@H]1c3c(cc(O)c(CC=C(C)C)c3OC)OC[C@@]21O ; COc1cc(O)cc2oc(=O)c3c4ccc(O)cc4oc3c12 ; CC(C)=CCc1c(O)ccc2c1oc(=O)c1c3ccc(O)cc3oc21]

The pterocarpan compound represented by Formula 1 of the present invention can be used as the form of a pharmaceutically acceptable salt, in which the salt is preferably acid addition salt formed by pharmaceutically acceptable free acids. The acid addition salt can be obtained from inorganic acids such as hydrochloric acid, nitric acid, phosphoric acid, sulfuric acid, hydrobromic acid, hydroiodic acid, nitrous acid and phosphorous acid, or non-toxic organic acids such as aliphatic mono/dicarboxylate, phenyl-substituted alkanoate, hydroxy alkanoate, alkandioate, aromatic acids and aliphatic/aromatic sulfonic acids. The pharmaceutically non-toxic salts are exemplified by sulfate, pyrosulfate, bisulfate, sulphite, bisulphite, nitrate, phosphate, monohydrogen phosphate, dihydrogen phosphate, metaphosphate, pyrophosphate, chloride, bromide, iodide, fluoride, acetate, propionate, decanoate, caprylate, acrylate, formate, isobutylate, caprate, heptanoate, propiolate, oxalate, malonate, succinate, suberate, cabacate, fumarate, maliate, butyne-1,4-dioate, hexane-1,6-dioate, benzoate, chlorobenzoate, methylbenzoate, dinitrobenzoate, hydroxybenzoate, methoxybenzoate, phthalate, terephthalate, benzenesulfonate, toluenesulfonate, chlorobenzenesulfonate, xylenesulfonate, phenylacetate, phenylpropionate, phenylbutylate, citrate, lactate, hydroxybutylate, glycolate, malate, tartrate, methanesulfonate, propanesulfonate, naphthalene-1-sulfonate, naphthalene-2-sulfonate and mandelate.

The acid addition salt in this invention can be prepared by the conventional method known to those in the art. For example, the pterocarpan compound represented by Formula 1 is dissolved in excessive acid aqueous solution, followed by salt precipitation using water-miscible organic solvent such as methanol, ethanol, acetone, or acetonitrile.

Equal amount of the pterocarpan compound represented by Formula 1 and acid aqueous solution or alcohol are heated, followed by drying the mixture to give acid addition salt or suction-filtering the precipitated salt to give the same.

A pharmaceutically acceptable metal salt can be prepared by using base. Alkali metal or alkali earth metal salt is obtained by the following processes: dissolving the compound in excessive alkali metal hydroxide or alkali earth metal hydroxide solution; filtering non-soluble compound salt; evaporating the remaining solution and drying thereof. At this time, the metal salt is preferably prepared in the pharmaceutically suitable form of sodium, potassium, or calcium salt. And the corresponding silver salt is prepared by the reaction of alkali metal or alkali earth metal salt with proper silver salt (ex; silver nitrate).

This invention includes not only the pterocarpan compound represented by Formula 1 but also the pharmaceutically acceptable salts thereof, solvates, hydrates, racemates, or isomers.

The pterocarpan compound represented by Formula 1 of the present invention can be prepared by the following methods, but not always limited thereto.

### Preparation Method 1

The preparation method of the pterocarpan compound represented by Formula 1 of the present invention is composed of the following steps:

obtaining soybean leaf extracts by adding soybean leaves to water, C1-C4 alcohol or the mixture thereof (step 1);

obtaining fractions by solvent partitioning the soybean leaf extracts obtained in step 1, by using water, dichloromethane, and ethylacetate stepwise (step 2); and

performing column chromatography with the ethylacetate fraction obtained in step 2 by using organic solvents and then isolating and purifying the compound obtained thereby (step 3).

Hereinafter, the preparation method of the present invention is described in more detail step by step.

In step 1 of the preparation Method 1, soybean leaf extracts are obtained by adding soybean leaves to water, C1-C4 alcohol or the mixture thereof.

In this step, soybean leaf extracts are obtained as follows: the dried soybean leaves are finely chopped, which are loaded in glassware. Water, C1-C4 alcohol, or the mixture thereof is added thereto, followed by extraction with raising temperature or at room temperature. To increase the extraction efficiency, the above procedure can be repeated several times. Then, the extracts stands for a while, followed by filtering with filter paper to give methanol extracts.

In step 2, fractions are obtained by fractioning the soybean leaf extracts obtained in step 1 by using water, dichloromethane, and ethylacetate stepwise.

Particularly, the soybean leaf extracts obtained in step 1 were suspended in water, followed by fractionation using dichloromethane and ethylacetate stepwise as the extraction solvents to give dichloromethane fraction, ethylacetate fraction and water fraction.

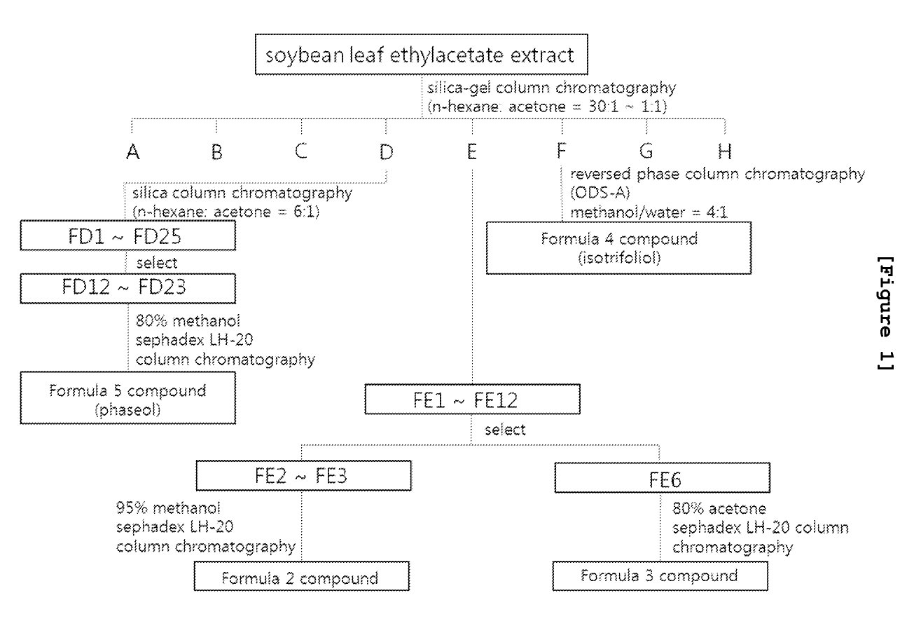

Step 3 is to separate and purify the compound from the ethylacetate fraction obtained in step 2 through silica-gel column chromatography using organic solvents as the elution solvents (see FIG. 1).

Particularly, the ethylacetate fraction, among all the fractions obtained in step 2, proceeded to silica-gel column chromatography using hexane/acetone (30:1-1:1) as the elution solvents, resulting in 8 fractions (A-H). Among the fractions, fraction E (hexane/acetone=8:1) proceeded to silica-gel column chromatography using chloroform/ethylacetate (10:1) as the elution solvent to give 12 fractions (FE1-FE12). Again among these 12 fractions, FE2 and FE3 were combined, which proceeded to Sephadex LH-20 column chromatography using 95% methanol as the elution solvent, by which the novel pterocarpan compound represented by Formula 2 was isolated.

Among the 12 fractions, FE6 was also selected and proceeded to Sephadex LH-20 column chromatography using 80% acetone as the elution solvent, and as a result, the novel pterocarpan compound represented by Formula 3 was isolated.

Further, the fraction F (hexane/acetone=6:1) proceeded to reversed phase (ODS-A) column chromatography using methanol/water (4:1) as the elution solvent, and as a result, the novel compound presented by Formula 4 (isotripoliol) was isolated.

The fraction D (hexane:acetone=10:1) proceeded to silica-gel column chromatography using hexane/acetone (6:1) as the elution solvent to give 25 fractions (FD1-FD 25). Among these 25 fractions, FD12-FD23 were combined, which proceeded to Sephadex LH-20 column chromatography using 80% methanol as the elution solvent, and as a result, the novel compound represented by Formula 5 (phaseol) was isolated.

### Preparation Method 2

The preparation method of the pterocarpan compound represented by Formula 1 of the present invention contains the following steps:

obtaining soybean leaf ethylacetate extracts by adding ethylacetate to soybean leaves (step 1); and

performing column chromatography with the soybean leaf ethylacetate extracts obtained in step 1 by using hexane/acetone as the elution solvent and then separating and purifying the compound obtained thereby (step 2).

Hereinafter, the preparation method of the present invention is described in more detail step by step.

In step 1 of the preparation Method 2, soybean leaves are extracted by using ethylacetate.

In this step, soybean leaf ethylacetate extracts are obtained as follows: the dried soybean leaves are finely chopped, which are loaded in glassware. Ethylacetate is added thereto, followed by extraction at room temperature. To increase the extraction efficiency, the above procedure can be repeated several times. Then, the extracts stand for a while, followed by filtering with filter paper to give ethylacetate extracts.

In step 2, silica-gel column chromatography is performed with the ethylacetate extracts obtained in step 1 by using organic solvents as the elution solvents to separate and purify the target compound.

This step can be performed by the same manner as described in step 3 of preparation Method 1.

The present invention also provides a pharmaceutical composition for the prevention or treatment of metabolic disease or complications thereof containing the pterocarpan compound represented by Formula 1 or the pharmaceutically acceptable salt thereof as an active ingredient.

To verify the activity targeting metabolic disease of the pterocarpan compound represented by Formula 1, the inhibitory activities of the formula 1 compounds on α-glucosidase, hACAT-1 and -2, and LDL-oxidation were measured first.

Particularly, α-glucosidase inhibiting activities of pterocarpan compounds included in the composition of the present invention were measured by the method of Kato et al (J. Med. Chem., 2005, 48: 2036-2044) with a slight modification. As a result, the pterocarpan compound of the present invention demonstrated IC₅₀ (the inhibition concentration by 50%) values of α-glucosidase to be 6.0-112.0 μM. In particular, the IC₅₀ values of compounds represented by Formula 4 and Formula 5 were 23.0 μM and 6.0 μM, respectively, indicating that they had high α-glucosidase inhibiting activity (see Table 1).

In the meantime, hACAT-1 and hACAT-2 inhibitory activities of compounds of the present invention were measured. As a result, the pterocarpan compounds represented by Formula 2-Formula 5 of the present invention inhibited hACAT-1 and hACAT-2 activities 14.9-85.9% and 7.9-67.4%, respectively, at 100 μM. In particular, the compound represented by Formula 5 (phaseol) showed potent hACAT-1 and hACAT-2 inhibitory activities reaching respectively 85.8% and 67.4% at 100 μM (see Table 2).

Further, LDL-oxidation inhibitory activities of compounds of the present invention were measured. The compounds represented by Formula 2-Formula 5 of the present invention showed anti-oxidation activities with IC₅₀ values to be 1.0-51.8 μM. In particular, the compounds represented by Formula 4 and Formula 5 demonstrated strong inhibitory activities on LDL-oxidation with IC₅₀ of 4.5 μM and 1.0 μM, respectively (see Table 3).

The pterocarpan compound of the present invention was orally administered to mice for toxicity test. As a result, the estimated LD₅₀ values of pterocarpan compound was much greater than 1,000 mg/kg in mice. The pterocarpan compound orally administered in this experiment was evaluated to be a safe substance (see Experimental Example 4).

Combining all the results above together, the pterocarpan compounds of the present invention inhibited efficiently α-glucosidase, human acyl-CoA: cholesterol acyltransferase-1 and -2 (hACAT-1 and hACAT-2) activities, and LDL oxidation activities as well but hardly has toxicity, so that the pterocarpan compounds of the present invention can be effectively used as a pharmaceutical composition for the prevention or treatment of metabolic disease or complications thereof.

The metabolic disease in this invention is selected from the group consisting of diabetes, hyperlipidemia, atherosclerosis, fatty liver, obesity, and metabolic syndrome. The complications of metabolic disease in this invention are selected from the group consisting of coronary artery disease, angina pectoris, carotid artery disease, stroke, cerebral arteriosclerosis, hypercholesterolemia, cholesterol gallstone, hypertriglyceridemia, hypertension, cataract, renal disease, neurological disorder, chronic inflammatory disease, and infectious disease.

The pterocarpan compounds of the present invention can be administered alone or treated together with surgical operation, radio-therapy, hormone therapy, chemo-therapy, and biological regulators in order to prevent or treat metabolic disease such as diabetes, hyperlipidemia, atherosclerosis, fatty liver, obesity, or metabolic syndrome, or complications thereof.

Further, the composition of the present invention can contain additionally one or more active ingredients having the same or similar functions to the pterocarpan compound isolated from soybean leaves.

In addition, the present invention also provides a treatment method for metabolic disease or complications thereof containing the step of administering a therapeutically effective dose of the pterocarpan compound represented by Formula 1 or the pharmaceutically acceptable salt thereof to a patient in need of treatment.

The term “prevention” used in this invention indicates all the actions that can suppress or delay the development of disease by administering the composition. In this invention, “improvement” or “treatment” indicates all the actions that can improve or relieve the symptoms of disease or that can induce any advantageous changes in a patient.

The term “administration” in this invention indicates providing a material to a patient via a proper method. The administration pathway in this invention includes every possible pathway as long as the composition of the present invention can be delivered to the target tissues through it, which includes oral and parenteral administration pathways. At this time, the composition can be administered by using a proper device that helps an active material to reach the target cells.

The composition of the present invention can be prepared for oral or parenteral administration by mixing with generally used diluents or excipients such as fillers, extenders, binders, wetting agents, disintegrating agents and surfactants.

Solid formulations for oral administration are tablets, pills, powders, granules, capsules, and troches. These solid formulations are prepared by mixing the compound of the present invention with one or more suitable excipients such as starch, calcium carbonate, sucrose or lactose, gelatin, etc. Except for the simple excipients, lubricants, for example magnesium stearate, talc, etc, can be used. Liquid formulations for oral administrations are suspensions, solutions, emulsions and syrups, and the above-mentioned formulations can contain various excipients such as wetting agents, sweeteners, aromatics and preservatives in addition to generally used simple diluents such as water and liquid paraffin.

Formulations for parenteral administration are sterilized aqueous solutions, water-insoluble excipients, suspensions, emulsions, lyophilized preparations and suppositories.

Water insoluble excipients and suspensions can contain, in addition to the active compound or compounds, propylene glycol, polyethylene glycol, vegetable oil like olive oil, injectable ester like ethylolate, etc. Suppositories can contain, in addition to the active compound or compounds, witepsol, macrogol, tween 61, cacao butter, laurin butter, glycerol, gelatin, etc.

The composition of the present invention is administered by a pharmaceutically effective dose. In this invention, the “pharmaceutically effective dose” indicates the medicinally applicable amount enough to treat disease at a reasonable benefit/risk ratio. The effective dose can be determined by considering the type and severity of disease, activity of drug, patient's sensitivity to drug, administration time, administration pathway and excretion rate, treatment period, other drugs co-treated, and other factors known in the field of medicine. The composition of the present invention can be administered alone or treated together with other therapeutic agents. Considering all the factors that might affect, it is important to administer the most effective dose meaning the minimum amount but with maximum effect without side effects, which can be easily determined by those in the art.

Particularly, the effective dose of the compound of the present invention can be adjusted by the age, gender, and body weight of patient, which is generally 0.1-100 mg/kg, and more preferably 0.5-10 mg/kg. The compound can be administered every day or every other day, once a day—three times a day. Again, the dose can be reduced or increased according to the administration pathway, the level of obesity, gender, body weight, age, etc, so that the present invention cannot be limited thereto in any way.

The present invention also provides a health functional food composition for the prevention or improvement of metabolic disease or complications thereof containing the pterocarpan compound represented by formula 1 or the pharmaceutically acceptable salt thereof as an active ingredient.

The compounds of the present invention inhibit the activities of α-glucosidase and hACAT-1 and hACAT-2 involved in metabolic disease and has potent LDL oxidation inhibitory activity (see Table 1-Table 3), so the pterocarpan compound of the present invention can be added to any health functional food including food and beverages for the purpose of preventing or improving metabolic disease or complications thereof.

In the health functional food composition of the present invention, the metabolic disease in this invention is selected from the group consisting of diabetes, hyperlipidemia, atherosclerosis, fatty liver, obesity, and metabolic syndrome. The complications of metabolic disease in this invention are selected from the group consisting of coronary artery disease, angina pectoris, carotid artery disease, stroke, cerebral arteriosclerosis, hypercholesterolemia, cholesterol gallstone, hypertriglyceridemia, hypertension, cataract, renal disease, neurological disorder, chronic inflammatory disease, and infectious disease.

In the health functional food composition of the present invention, the food added with the pterocarpan compound represented by Formula 1 is not limited. For example, the compound of the present invention can be added to drinks, meat, sausages, bread, biscuits, chocolates, candies, snacks, cookies, pizza, ramyuns, flour products, gums, dairy products including ice cream, soups, beverages, tea, alcohol drinks and vitamin complex, etc, and in wide sense, almost every food applicable in the production of health food can be included.

The pterocarpan compound of the present invention can be added to food as it is or as mixed with other food components according to the conventional method. The mixing ratio of active ingredients can be regulated according to the purpose of use (prevention or health enhancement). In general, to produce health functional food, the compound of the present invention is added preferably by 0.1-90 weight part. However, if long term administration is required for health and hygiene or regulating health condition, the content can be lower than the above but higher content can be accepted as well since the compound of the present invention has been proved to be very safe.

The composition for health beverages of the present invention can additionally include various flavors or natural carbohydrates, etc, like other beverages. Besides, natural sweetening agents (thaumatin, stevia extracts, for example rebaudioside A, glycyrrhizin, etc.) and synthetic sweetening agents (saccharin, aspartame, etc.) can be included as a sweetening agent. The content of the natural carbohydrate is preferably 1-20 g, more preferably 5-10 g, in 100 g of the composition.

In addition to the ingredients mentioned above, the health functional food composition of the present invention can include in a variety of nutrients, vitamins, minerals (electrolytes), flavors including natural flavors and synthetic flavors, coloring agents and extenders (cheese, chocolate, etc.), pectic acid and its salts, alginic acid and its salts, organic acid, protective colloidal viscosifiers, pH regulators, stabilizers, antiseptics, glycerin, alcohols, carbonators which used to be added to soda, etc. The composition of the present invention can also include natural fruit juice, fruit beverages, and fruit flesh addable to vegetable beverages.

All the mentioned ingredients can be added singly or together. The mixing ratio of those ingredients does not matter in fact, but in general, each can be added by 0.1-20 weight part per 100 weight part of the pterocarpan compound of the present invention.

In addition, the present invention provides an anti-oxidative pharmaceutical composition, a health functional food composition, a cosmetic composition, or a feed additive, containing the pterocarpan compound represented by formula 1 or the pharmaceutically acceptable salt thereof as an active ingredient.

Owing to the potent anti-oxidative activity (see Table 3), the pterocarpan compound of the present invention can be effectively used for the prevention, treatment, or improvement of cancer, aging, coronary atherosclerosis, diabetes, arthritis, epilepsy, stroke, Parkinson's disease, Alzheimer's disease, autoimmune disease, and neurodegenerative disease.

The pterocarpan compound of the present invention can delay skin aging resulted from the oxidation of biomaterials, so that it can also be used as a functional cosmetic composition.

In addition, when the pterocarpan compound of the present invention is added to feeds as a feed additive, it not only helps to prevent acidification of feeds, but also helps the secretion of digestive enzymes in the pancreas and acts as an antioxidant therein when animals eat the feeds, so that the compound can prevent disease caused by active oxygen. When the processed meat product is produced from such animals that have been taking the said natural compound, quality of the meat product can be improved because oxidation in the meat has been prevented.

The anti-oxidative cosmetic composition of the present invention includes lotion, ointment, gel, cream, patch, or spray, but not always limited thereto. For the preparation of the anti-oxidative cosmetic composition of the present invention, the pterocarpan compound can be added by 1-15 weight part, preferably 2-10 weight part to the external composition for skin care. The external composition for skin care can additionally include a supplement generally used in the field of skin science such as fatty substance, organic solvent, resolvent, concentrate, gelling agent, softener, antioxidant, suspending agent, stabilizer, foaming agent, odorant, surfactant, water, ionic or non-ionic emulsifying agent, filler, sequestering agent, chelating agent, preserving agent, vitamin, blocker, moisturizing agent, essential oil, dye, pigment, hydrophilic or hydrophobic activator, lipid vesicle or other components generally used in a preparation for skin external application. The amount of the above supplement can be determined as generally accepted in the field of skin science.

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Isolation of Pterocarpan Compound from Soybean Leaves

Step 1: Preparation of Soybean Leaf Extracts

The pterocarpan compound included in the composition of the present invention is preferably obtained from soybean leaves (Glycine max leaves), but not always limited thereto. The said soybean leaves can be obtained from cultivation or purchased.

Herein, soybean was seeded in Jinju city, Gyeongsangnam-do, Korea. After 110 days of growing, soybean leaves were collected and dried in the shade. The dried soybean leaves (4 kg) were chopped, to which 12 L of ethylacetate was added. Extraction was performed at room temperature for 7 days. Ethylacetate soluble fraction was recovered by using filter paper, followed by concentration under the reduced pressure to give 76 g of ethylacetate extracts.

Step 2: Isolation and Identification of Pterocarpan Compounds

Step 2a: Preparation of the Novel Pterocarpan Compounds Represented by Formula 2 and Formula 3

The ethylacetate extracts obtained in step 1 proceeded to silica-gel column chromatography using hexane-acetone mixed solution (hexane:acetone=30:1 (4 L)→20:1 (2 L)→10:1 (2 L)→8:1 (2 L)→6:1 (2 L)→3:1 (2 L)→1:1 (1 L)) to give 8 fractions (A-H).

The fraction E (hexane:acetone=8:1, 4.6 g) proceeded to silica-gel column chromatography using chloroform:ethylacetate (10:1) mixed gradient solution to give 12 fractions (FE1-FE12). The fractions FE2 and FE3 were combined and proceeded to Sephadex LH-20 (Pharmacia Biotech AB, Uppsala, Sweden) column chromatography using 95% methanol as the elution solvent to give the novel compound represented by Formula 2 (8 mg).

In the meantime, the fraction FE6 proceeded to Sephadex LH-20 (Pharmacia Biotech AB, Uppsala, Sweden) column chromatography using 80% acetone as the elution solvent to give the novel compound represented by Formula 3 (5 mg).

NMR and mass spectrometry were performed to analyze the structures of those compounds. The results are shown below.

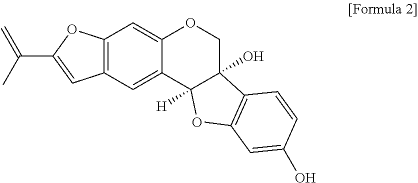 [SMILES: C=C(C)c1cc2cc3c(cc2o1)OC[C@@]1(O)c2ccc(O)cc2O[C@@H]31]

Chemical name: (6aS,11aS)-2-(pro-1-pene-2-yl)-6a,11a-dihydro-6H-benzofuro[3,2-c]furo[3,2-g]chromene-6a,9-diol;

Melting point (m.p.): 149-151° C.;

Optical rotation: [α]_{D}²⁰-14.2 (c 0.1, CH₃OH);

EIMS (m/z): 336[M]⁺;

HREIMS (m/z): 336.1007 (calcd for C₂₀H₁₆O₅ 336.0998);

CD(DMSO): λmax λε nm+166.7 (289), −57.6 (235);

¹H NMR (500 MHz, CD₃OD) δ 2.00 (3H, s, H-16), 3.90 (1H, d, J=11.4 Hz, H-6a), 4.07 (1H, d, J=11.4 Hz, H-6), 5.04 (1H, s, H-15), 5.27 (1H, s, H-11a), 5.58 (1H, s, H-15a), 6.14 (1H, s, H-10), 6.31 (1H, dd, J=8.1, 1.5 Hz, H-8), 6.61 (1H, s, H-12), 6.86 (1H, s, H-4), 7.09 (1H, d, J=8.1 Hz, H-7), 7.55 (1H, s, H-1);

¹³C NMR (125 MHz, CD₃OD) δ 19.8 (C-16), 72.0 (C-6), 77.9 (C-6a), 87.1 (C-11a), 99.3 (C-10), 100.3 (C-4), 104.1 (C-12), 109.9 (C-8), 113.5 (C-15), 118.9 (C-11b), 121.5 (C-6b), 124.8 (C-1), 125.7 (C-7), 125.9 (C-2), 134.6 (C-14), 155.2 (C-4-a), 157.3 (C-3), 158.9 (C-13), 161.6 (C-9), 162.7 (C-10a).

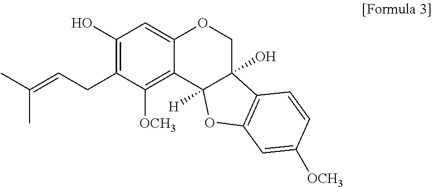 [SMILES: COc1ccc2c(c1)O[C@H]1c3c(cc(O)c(CC=C(C)C)c3OC)OC[C@@]21O]

Chemical name: (6aS,11aS)-1,9-dimethoxy-2-(3-methylbu-2-tene-1-yl))-6a,11a-dihydro-6H-benzofuro[3,2-c]chromene-3,6a-diol;

Melting point (m.p.): 137-140° C.;

Optical rotation: [α]_{D}²⁰-192 (c 0.1, CH₃OH);

EIMS (m/z): 384 [M]⁺;

HREIMS (m/z): 384.1572 (calcd for C₂₀H₁₆O₅ 384.1573);

¹H NMR (500 MHz, CD₃OD) δ 1.67 (3H, s, H-5′), 1.75 (3H, s, H-4′), 3.27 (2H, m, H-1′), 3.75 (OCH₃), 3.86 (1H, d, J=11.3 Hz, H-6a), 3.90 (OCH₃), 4.05 (1H, d, J=11.3 Hz, H-6), 5.15 (1H, m), 5.33 (1H, s, H-11a), 6.25 (1H, d, J=2.0 Hz, H-10), 6.30 (1H, s, H-4), 6.40 (1H, dd, J=8.1, 2.0 Hz, H-8), 7.15 (1H, d, J=8.1 Hz, H-7);

¹³C NMR (125 MHz, CD₃OD) δ 18.4 (C-5′), 24.1 (C-1′), 26.3 (C-4′), 56.5 (C-9), 63.8 (C-1), 71.4 (C-6), 77.3 (C-6a), 83.3 (C-11a), 97.3 (C-4), 99.4 (C-10), 108.5 (C-11b), 109.7 (C-8), 118.8 (C-2), 121.7 (C-6b), 125.2 (C-2′), 125.4 (C-7), 132.1 (C-3′), 156.6 (C-4-a), 161.3 (C-3), 161.5 (C-9), 161.7 (C-1), 162.5 (C-10a).

Step 2B: Preparation of the Compounds Represented by Formula 4 and Formula 5

The fraction F obtained in step 2A (hexane:acetone=6:1, 4.1 g) proceeded to reversed phase column chromatography (ODS-A, 12 nm, S-150 μM, eluate methanol:water=4:1) to give the compound represented by Formula 4 (20 mg).

The fraction D obtained in step 2A (hexane:acetone=10:1, 3.1 g) proceeded to silica-gel column chromatography using hexane:acetone (6:1) mixed gradient solution to give 25 fractions (FD1-FD25).

Among those fractions, FD12-FD23 containing a high concentration of the compound represented by Formula 5 were combined, followed by Sephadex LH-20 (Pharmacia Biotech AB, Uppsala, Sweden) using 80% methanol as the elution solvent. As a result, the compound represented by formula 5 was obtained (42 mg).

NMR and mass spectrometry were performed to analyze the structures of those compounds. The results are shown below.

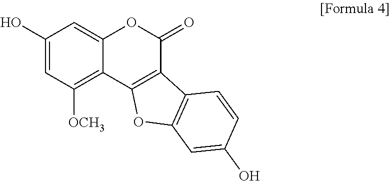 [SMILES: COc1cc(O)cc2oc(=O)c3c4ccc(O)cc4oc3c12]

Chemical name: isotrifoliol

Melting point (m.p.): >300° C.;

EIMS (m/z): 298 [M]⁺;

HREIMS (m/z): 298.0476 (calcd for C₁₆H₁₀O₆ 298.0477);

¹H NMR: (500 MHz, DMSO-d₆) δ 3.98 (OCH₃), 6.51 (1H, s, H-2), 6.52 (1H, s, H-4), 6.94 (1H, d, J=8.4 Hz, H-8), 7.13 (1H, s, H-10), 7.68 (1H, d, J=8.4 Hz, H-7);

¹³C NMR: (125 MHz, DMSO-d₆) δ 56.7 (C-1), 96.2 (C-4), 96.5 (C-2), 98.8 (C-10), 101.7 (C-6a), 114.2 (C-8), 114.6 (C-6b), 114.6 (C-11b), 120.6 (C-7), 155.7 (C-6), 156.2 (C-4-a), 157.0 (C-9), 157.1 (C-10a), 159.6 (C-3), 162.1 (C-11a).

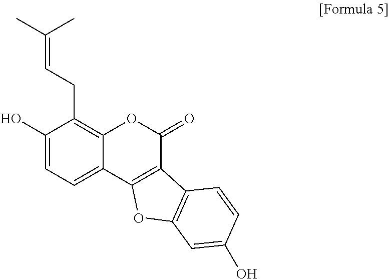 [SMILES: CC(C)=CCc1c(O)ccc2c1oc(=O)c1c3ccc(O)cc3oc21]

Chemical name: phaseol

Melting point (m.p.): 248-250° C.;

EIMS (m/z): 336 [M]⁺;

HREIMS (m/z): 336.1004 (calcd for C₂₀H₁₆O₅ 336.0998);

¹H NMR: (500 MHz, DMSO-d₆) δ 1.63 (3H, s, H-5′), 1.82 (3H, s, H-4′), 3.46 (2H, d, J=7.1 Hz, H-1′), 5.22 (1H, t, J=7.2, 6.3 Hz, H-2′), 6.94 (1H, d, J=8.4 Hz, H-8), 6.98 (1H, d, J=8.6 Hz, H-2), 7.15 (1H, s, H-10), 7.68 (1H, s, H-7), 7.70 (1H, s, H-1);

¹³C NMR: (125 MHz, DMSO-d₆) δ 18.2 (C-5′), 25.9 (C-4′), 99.0 (C-10), 102.1 (C-6a), 104.6 (C-11b), 113.2 (C-2), 114.3 (C-8), 114.9 (C-6b), 116.0 (C-4), 119.9 (C-7), 120.9 (C-1), 121.8 (C-1′), 132.0 (C-3′), 152.6 (C-4-a), 156.4 (C-10a), 157.3 (C-9), 157.9 (C-6), 159.0 (C-3), 160.2 (C-11a).

### Experimental Example 1

### Measurement of α-glucosidase Inhibitory Activity of Pterocarpan Compound

Following experiment was performed to investigate the effect of the pterocarpan compound of the present invention on α-glucosidase activity that is essential for carbohydrate metabolism.

Alpha-glucosidase inhibitory activity was measured by the nitrophenol method proposed by Kato et al. (J. Med. Chem., 2005, 48: 2036-2044) with slight modification. Particularly, the accumulation of chromophore generated by hydrolyzing the substrate p-nitrophenyl-α-D-glucopyranoside (Sigma-Aldrich) by α-glucosidase (EC 3.2.1.20, Baker Yeast) was measured with absorbance.

Fifty (50) μl of buffer (70 mM calcium phosphate, pH 6.8), 50 μl of the sample compound dissolved in 50% DMSO, 50 μl of α-glucosidase (0.1 unit/ml), and a substrate (5 mM, p-nitrophenyl-α-D-glucopyranoside) were added into each well of a 97-well plate (NUNC™), followed by reaction at 37° C. for 30 minutes. The reaction was terminated by adding 2 M NaOH. The amount of chromophore generated was measured at 405 nm using a microplate reader (ANTHOS™), based on which the inhibitory activity was calculated. The results are shown in Table 1.

TABLE 1

| | α-Glucosidase inhibitory |
|---|---|
| Pterocarpan | activity |
| compounds | (IC₅₀, μM) |
| Formula 2 | 90.4 |
| Formula 3 | 112.0 |
| Formula 4 | 23.0 |
| Formula 5 | 6.0 |

As shown in Table 1, the pterocarpan compound of the present invention demonstrated potent α-glucosidase inhibitory activity with showing IC₅₀ values of 6.0-112.0 μM. Particularly, the IC₅₀ values of compounds represented by Formula 4 and Formula 5 were 23.0 μM and 6.0 μM, respectively, indicating that they had high α-glucosidase inhibiting activity.

Therefore, the pterocarpan compounds represented by Formula 2-Formula 5 of the present invention were confirmed as α-glucosidase inhibitors, and they can be effectively used as compounds for the prevention or treatment of diabetes (Eur. J. Org. Chem., 5: 967, 2001), obesity (Int. J. Obes., 1987, 11(Supple 2): 28), viral disease (Diabetes Care, 2005, 28: 154), cancer (Cancer Commun., 1989, 1: 373; cancer Res., 1986, 46: 5315), and other diseases caused by metabolism imbalance, and complications thereof.

### Experimental Example 2

### Measurement of hACAT Inhibitory Activity of Pterocarpan Compound

Following experiment was performed to investigate the effect of the pterocarpan compounds of the present invention on hACAT-1 and hACAT-2 that play a key role in accelerating intracellular cholesterol accumulation by converting cholesterol into cholesteryl ester.

Step 1: Protein Recombination

cDNA of each hACAT-1 and hACAT-2 obtained by human liver cDNA library screening was inserted into baculovirus vector, which was then introduced into the insect cell sf9 to produce target virus. The recombinant virus of each hACAT-1 and hACAT-2 was separated by plaque purification and then amplified three times to increase titer of viral stock.

Hi5 insect cells demonstrating high protein expression efficiency were infected with the recombinant virus (multiplicity of infection: 1), followed by shaking-culture at 27° C. for one day. To separate microsome fractions from the cultured Hi5 cells over-expressing hACAT-1 and hACAT-2, centrifugation was performed at 500 rpm for 15 minutes. The collected cells were lysed by quick-freezing/quick-thawing in hypotonic buffer, followed by ultracentrifugation at 100,000 rpm for one hour. The obtained microsome fractions were suspended in hypotonic buffer to make the protein concentration to be 8 mg/ml, which was stored in −70° C. freezer until use.

Step 2: Measurement of hACAT Activity

Human ACAT (hACAT) activity was measured by the method of Brecher & Chan (P. Brecher and C. Chan, Biochem. Biophys. Acta, 1980, 617: 458) with slight modification using [1⁻¹⁴C] oleoyl-CoA (56.0 μCi/μmol; Amersham) as a substrate. 10 μl of each of the pterocarpan compounds represented by Formula 2-Formula 5 obtained in Example 1 in dimethylsulfoxide (DMSO) was mixed with 4.0 μl of the microsome solution obtained in step 1, 20.0 μl of assay buffer (0.5 M KH₂PO₄, 10 mM DTT, pH 7.4; Sigma), 15.0 μl of bovine serum albumin (BSA, stock solution conc. 40 mg/ml; Sigma), 2.0 μl of cholesterol (stock solution conc. 20 mg/ml; Sigma), and 41.0 μl of distilled water, followed by pre-reaction at 37° C. for 15 minutes. In this assay, the negative control was used the DMSO alone, and the positive control was used a known ACAT inhibitor, oleic acid anilide.

Eight (8) μl of [1⁻¹⁴C] oleoyl-CoA (0.05 μCi, final conc. 10.0 μM) was added to the above reaction mixture, followed by reaction at 37° C. for 30 minutes. The reaction was terminated by adding 1 ml of isopropanol:heptane mixed solution (4:1 (v/v)). Six hundred (600) μl of heptane and 200 μl of 0.1 M KH₂PO₄ (pH 7.4) were added thereto and the reaction mixture was vortexed vigorously and centrifuged at 300 rpm for 5 minutes. The obtained upper phase (100 μl) was added in a scintillation vial, to which 4 ml of scintillation solution (Lipoluma, Lumac Co.) were added. Radioactivity of the upper phase was measured by 1450 Microbeta liquid scintillation counter (Wallacoy).

hACAT activity was calculated based on the radioactivity, the amount of synthesized cholesteryl oleated, measured above and expressed as a difined unit, pico mole per 1 mg of protein for 1 minute (pico mole/minute/mg protein). The results are shown in Table 2.

TABLE 2

| | |
|---|---|
| | Inhibitory activity |
| Pterocarpan | at 100 μM (%) |

| compound | hACAT1 | hACAT2 |
|---|---|---|
| Formula 2 | 29.7 | 22.9 |
| Formula 3 | 35.2 | 20.3 |
| Formula 4 | 14.9 | 7.9 |
| Formula 5 | 85.9 | 67.4 |

As shown in Table 2, the pterocarpan compounds represented by Formula 2-Formula 5 of the present invention were confirmed to have hACAT-1 and hACAT-2 inhibitory activities 14.9-85.9% and 7.9-67.4%, respectively, at 100 μM. In particular, the compound represented by Formula 5 (phaseol) showed potent hACAT-1 and hACAT-2 inhibitory activities reaching respectively by 85.8% and 67.4% at 100 μM. Therefore, the pterocarpan compound of the present invention has been confirmed to be effective in inhibiting ACAT activity that accelerates the accumulation of cholesterol, so that it can be effectively used for the prevention or treatment of cardiovascular disease caused by cholesteryl ester synthesis and accumulation such as hyperlipidemia, coronary artery disease, atheroosclerosis and myocardial infarction, or high fat diet induced obesity, and complications thereof.

### Experimental Example 3

### Measurement of LDL Anti-Oxidative Activity of Pterocarpan Compound

Following experiment was performed to investigate the LDL-oxidation inhibitory activity of the pterocarpan compound of the present invention.

Cu²⁺ has been known to induce LDL-oxidation (Cu²⁺-mediated LDL-oxidation). In this invention, dialdehyde, the oxide of unsaturated fatty acid, generated during LDL-oxidation was measured by TBA (thiobarbituric acid) method to investigate the anti-oxidative activity of the pterocarpan compound isolated from soybean leaves (Packer, L. Ed. (1994) Methods in Enzymology Vol. 234, Oxygen radicals in biological systems Part D. Academic press, San Diego).

Three hundred (300) ml of human plasma were centrifuged at 100,000×g for 24 hours to remove floating VLDL/chylomicron layer in upper part. The specific gravity of the remaining solution was adjusted to 1,063 g/ml, followed by re-centrifugation at 100,000×g for 24 hours, and 25 ml of the floating LDL (1.5-2.5 mg protein/ml) in upper layer was separated.

Twenty (20) μl of the separated LDL (protein conc. 50-100 μg/ml) was mixed with 210 μl of 10 mM PBS, to which 10 μl of each of the pterocarpan compounds represented by Formula 2-Formula 5 of the present invention was added respectively.

The pterocarpan compounds were dissolved in DMSO, which were further diluted properly for the experiment. In this assay, the negative control was used the DMSO only, while the positive control was used a known LDL-antioxidant, probucol.

Ten (10) μl of 0.25 mM CuSO₄ was added to the above reaction mixture, followed by reaction at 37° C. for 4 hours. The reaction was terminated by adding 1 ml of 20% trichloroacetic acid (TCA) solution. One (1) ml of 0.67% thiobarbituric acid (TBA) dissolved in 0.05 N NaOH solution was added thereto, followed by stirring for 10 seconds. Then, the reaction mixture was heated at 95° C. for 5 minutes to induce color development. The solution was cooled down with iced water and centrifuged at 3,000 rpm for 5 minutes to separate supernatant. The amount of malondialdehyde (MDA) generated by the color development was calculated by measuring optical density at 540 nm (OD₅₄₀) with UV-VIS Spectrophotometer.

PBS standard solution containing 0-10 nmol MDA was prepared by 250 μl with tetramethoxypropane malonaldehyde bis(dimethylacetal) stock solution. Color development was induced with the standard solution, followed by measuring OD₅₄₀. The generated MDA was quantified by using the standard curve in experiments with the pterocarpan compound of the present invention. The results are shown in Table 3.

TABLE 3

| | LDL-oxidation |
|---|---|
| Pterocarpan | inhibitory activity |
| compound | (IC₅₀, μM) |
| Formula 2 | 43.0 |
| Formula 3 | 51.8 |
| Formula 4 | 4.5 |
| Formula 5 | 1.0 |

As shown in Table 3, the pterocarpan compounds represented by Formula 2-Formula 5 of the present invention demonstrated strong anti-oxidative activity to LDL with showing IC₅₀ values of 1.0-51.8 μM. In particular, the compounds represented by Formula 4 and Formula 5 were confirmed to have higher LDL-oxidation inhibitory activities with IC₅₀ values of 4.5 μM and 1.0 μM, respectively.

Therefore, the pterocarpan compound of the present invention can be effectively used for the prevention or treatment of metabolic disease induced by oxidation of LDL such as diabetes, hyperlipidemia, atherosclerosis, fatty liver, obesity, and metabolic syndrome, and complications thereof.

### Experimental Example 4

### Acute Toxicity Test in ICR Mice Via Oral Administration

In order to investigate acute toxicity of the pterocarpan compound of the present invention, following experiments were performed in ICR mice.

Step 1: Preparation of ICR Mice

Four (4)-week old specific pathogen free ICR mice (12 female mice and 12 male mice, three of each assigned for each dose group) were used in this experiment. The mice were raised in an animal laboratory at 22±3° C., with the humidity of 55±10%, and under the light condition of 12L/12D. The mice were adapted for 1 week in an animal laboratory before being used. Pellet-type diet for test animals (CheilJedang, Co., for mice and rats) and water were sterilized and provided freely.

Step 2: Drug Administration

The pterocarpan compounds represented by Formula 2-Formula 5 obtained in Example 1 were dissolved in 0.5% tween at the concentration of 50 mg/ml, which were orally administered to each mouse at the dose of 0.2 ml per 20 g of mouse weight (500 mg/kg) or at the dose of 0.4 ml per 20 g of mouse weight (1,000 mg/kg).

The samples were administered orally just once. After the administration, side effects and death were observed for 7 days. Precisely, changes of any symptoms and death of an animal were observed 1, 4, 8, and 12 hours after the oral administration on the first day, and from the next day to the 7th day of administration, and once or more in the morning and once or more in the afternoon from second day through the 7th day. On the 7th day from the administration, the animals were sacrificed and anatomized. The internal organs were examined by the naked eye. Weight changes had been observed every 24 hours from the day of administration to investigate whether or not the pterocarpan compound could induce weight loss in those animals.

As a result, neither specific clinical symptoms nor death by the administration of the test sample were observed in those mice. In addition, no toxicity change was detected in mice either from the observation of weight changes, hematological tests, biochemical tests of blood, or autopsy. Therefore, the pterocarpan compounds represented by Formula 2-Formula 5 of the present invention isolated from soybean leaves were evaluated to be safe substances since they did not cause any toxic change in mice up to the level of 1000 mg/kg and their estimated LD₅₀ values are much greater than 1,000 mg/kg in mice.

Therefore, it was confirmed that the pterocarpan compound of the present invention inhibited α-glucosidase hACAT-1 and hACAT-2 activities efficiently, and had strong anti-oxidative activity on LDL-oxidation, so that it could be effectively used as a composition for the prevention or treatment of metabolic disease or complications thereof with less side effects.

In the meantime, the pterocarpan compounds represented by Formula 2-Formula 5 of the present invention can be formulated in many different forms to meet the purpose of use. Followings are the examples of formulations containing the compounds represented by Formula 2-Formula 5 as active ingredients and the methods for the formulation thereof, which cannot limit the present invention, though.

### Manufacturing Example 1

### Preparation of Pharmaceutical Formulations

### <1-1> Preparation of Powders

| | |
|---|---|
| Pterocarpan compound | 500 ng |
| Lactose | 1 g |

Powders were prepared by mixing all the above components, which were filled in airtight packs according to the conventional method for preparing powders.

### <1-2> Preparation of Tablets

| | |
|---|---|
| Pterocarpan compound | 500 ng |
| Corn starch | 100 mg |
| Lactose | 100 mg |
| Magnesium stearate | 2 mg |

Tablets were prepared by mixing all the above components by the conventional method for preparing tablets.

### <1-3> Preparation of Capsules

| | |
|---|---|
| Pterocarpan compound | 500 ng |
| Corn starch | 100 mg |
| Lactose | 100 mg |
| Magnesium stearate | 2 mg |

Capsules were prepared by mixing all the above components, which were filled in gelatin capsules according to the conventional method for preparing capsules.

### <1-4> Preparation of Injectable Solutions

| | |
|---|---|
| Pterocarpan compound | 500 ng |
| Mannitol | 180 mg |
| Na₂HPO₄•2H₂O | 26 mg |
| Distilled water | 2974 mg |

Injectable solutions were prepared by mixing all the above components by the conventional method for preparing injectable solutions.

### Manufacturing Example 2

### Preparation of Health Functional Food

### <2-1> Preparation of Health Functional Food

| Pterocarpan compound | 500 | ng |
|---|---|---|
| Vitamin complex | proper amount | |
| Vitamin A acetate | 70 | μg |
| Vitamin B6 | 0.5 | mg |
| Vitamin B12 | 0.2 | μg |
| Vitamin C | 10 | mg |
| Biotin | 10 | μg |
| Nicotinic acid amide | 1.7 | mg |
| Folic acid | 50 | mg |
| Calcium pantothenate | 0.5 | mg |
| Minerals | proper amount | |
| Ferrous sulfate | 1.75 | mg |
| Zinc oxide | 0.82 | mg |
| Magnesium carbonate | 25.3 | mg |
| Potassium phosphate monobasic | 15 | mg |
| Potassium phosphate dibasic | 55 | mg |
| Potassium citrate | 90 | mg |
| Calcium carbonate | 100 | mg |
| Magnesium chloride | 24.8 | mg |

Vitamins and minerals were mixed according to the preferable composition ratio for health functional food. However, the composition ratio can be adjusted. The constituents were mixed according to the conventional method for preparing health functional food and then the health functional food composition was prepared according to the conventional method.

### <2-2> Preparation of Health Beverage

| | | |
|---|---|---|
| Pterocarpan compound | 500 | ng |
| Citric acid | 1000 | mg |
| Oligosaccharide | 100 | g |
| Maesil (Prunus mume) Extract | 2 | g |
| Taurine | 1 | g |
| Purified water | up to 900 | ml |

The above constituents were mixed according to the conventional method for preparing health beverage. The mixture was heated at 85° C. for 1 hour with stirring and then filtered. The filtrate was loaded in 2 liter sterilized containers, which were sealed and sterilized again, stored in a refrigerator until they would be used for the preparation of a composition for health beverage.

The constituents appropriate for favorite beverage were mixed according to the preferred mixing ratio but the composition ratio can be adjusted according to regional and national preferences, etc.

### Manufacturing Example 3

### Preparation of Cosmetic Composition

### <3-1> Preparation of Cream

| | |
|---|---|
| Cetostearyl alcohol | 2.8 weight part |
| Wax | 2.6 weight part |
| Stearic acid | 1.4 weight part |
| Glyceryl monostearate, lipophilic | 2 weight part |
| PEG-100 stearate | 1 weight part |
| Phosphosorbitan sesquioleate | 1.4 weight part |
| Jojoba oil | 4 weight part |
| Squalane | 3.8 weight part |
| Polysorbate 60 | 1.1 weight part |
| Macadamia oil | 2 weight part |
| Tocopheryl acetate | 0.2 weight part |
| Methylpolysiloxane | 0.4 weight part |
| Ethylparaben | 0.1 weight part |
| Propyl paraben | 0.1 weight part |
| Euxyl K-400 | 0.1 weight part |
| 1,3-butyleneglycol | 7 weight part |
| Methylparaben | 0.05 weight part |
| Glycerine | 6 weight part |
| D-panthenol | 0.2 weight part |
| Pterocarpan compound | 4.6 weight part |
| Triethanolamine | 0.2 weight part |
| pt 41891 | 0.2 weight part |
| p-H₂O | 46.05 weight part |

### <3-2> Preparation of Cream

| | |
|---|---|
| Cetostearyl alcohol | 1.6 weight part |
| Stearic acid | 1.4 weight part |
| Glyceryl monostearate, lipophilic | 1.8 weight part |
| PEG-100 stearate | 2.6 weight part |
| Phosphosorbitan sesquioleate | 0.6 weight part |
| Squalene | 4.8 weight part |
| Macadamia oil | 2 weight part |
| Jojoba oil | 2 weight part |
| Tocopheryl acetate | 0.4 weight part |
| Methylpolysiloxane | 0.2 weight part |
| Ethylparaben | 0.1 weight part |
| Propyl paraben | 0.1 weight part |
| 1,3-butyleneglycol | 4 weight part |
| Methylparaben | 0.1 weight part |
| Xanthan gum | 0.1 weight part |
| Glycerine | 4 weight part |
| D-panthenol | 0.15 weight part |
| Allantoin | 0.1 weight part |
| Pterocarpan compound | 3.5 weight part |
| Carbomer (2% aq. Sol) | 4 weight part |
| Triethanolamine | 0.15 weight part |
| Ethanol | 3 weight part |
| pt 41891 | 0.1 weight part |
| p-H₂0 | 48.3 weight part |

### Manufacturing Example 4

### Preparation of Feed Additive

Feed additive comprising the Pterocarpan compound of the present invention as an active ingredient was prepared according to the composition shown below.

| | | |
|---|---|---|
| Pterocarpan compound | 0.1-20 | weight part |
| Lipase | 0.001-0.01 | weight part |
| Calcium phosphate, tribasic | 1-20 | weight part |
| Vitamin E | 0.01-0.1 | weight part |
| Enzyme powder | 1-10 | weight part |
| Lactic acid bacteria | 0.1-10 | weight part |
| Bacillus culture | 0.01-10 | weight part |
| Glucose | 20-90 | weight part |

What is claimed is:

1. A novel pterocarpan compound represented by Formula 1 or a pharmaceutically acceptable salt thereof: 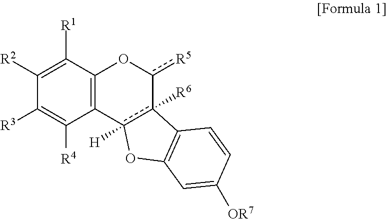
(In the Formula 1, [symbol]
is single bond or double bond;
R¹ is hydrogen or isobutenyl group;
R² is hydroxyl group, and R³ is hydrogen or isobutenyl group, or R² and R³ can form 5-7 membered heteroaryl ring with carbon atoms conjugated with them in addition to 1 heteroatom selected from the group consisting of N, O, and S. At this time, the heteroaryl ring is non-replaceable or can be replaced with isopropenyl group;
R⁴ is hydrogen or C₁-C₄ straight or branched alkoxy group;
R⁵ is hydrogen or ═O;
R⁶ is hydrogen or hydroxyl group; and
R⁷ is hydrogen or C₁-C₄ straight or branched alkyl group).

2. The novel pterocarpan compound according to claim 1, wherein the [symbol] is single bond or double bond;
R² is hydrogen or isobutenyl group;
R² is hydroxyl group, and R³ is hydrogen or isobutenyl group, or R² and R³ can form furan ring with carbon atoms conjugated with them, and at this time the furan ring is non-replaceable or can be replaced with isopropenyl group;
R⁴ is hydrogen or methoxy group;
R⁵ is hydrogen or ═O;
R⁶ is hydrogen or hydroxyl group; and
R⁷ is hydrogen or methyl group.

3. The novel pterocarpan compound according to claim 1, wherein the pterocarpan compound represented by Formula 1 is one or more selected from the group consisting of the compounds represented by Formula 2-Formula 5:

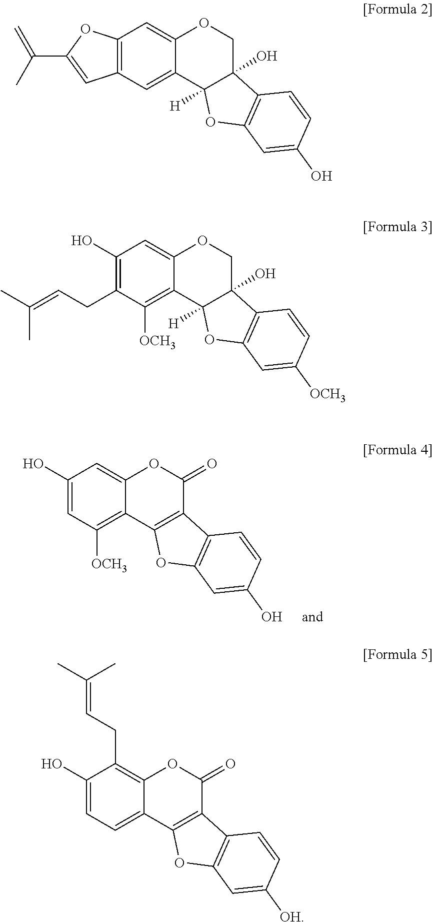 [SMILES: C=C(C)c1cc2cc3c(cc2o1)OC[C@@]1(O)c2ccc(O)cc2O[C@@H]31 ; COc1ccc2c(c1)O[C@H]1c3c(cc(O)c(CC=C(C)C)c3OC)OC[C@@]21O ; COc1cc(O)cc2oc(=O)c3c4ccc(O)cc4oc3c12 ; CC(C)=CCc1c(O)ccc2c1oc(=O)c1c3ccc(O)cc3oc21]

4. A pharmaceutical composition for the prevention or treatment of metabolic disease or complications thereof containing the pterocarpan compound represented by Formula 1 or the pharmaceutically acceptable salt thereof of claim 1 as an active ingredient.

5. The pharmaceutical composition for the prevention or treatment of metabolic disease of complications thereof according to claim 4, wherein the metabolic disease is selected from the group consisting of diabetes, hyperlipidemia, atherosclerosis, fatty liver, and obesity; and the complication is selected from the group consisting of coronary artery disease, angina pectoris, carotid artery disease, stroke, cerebral arteriosclerosis, hypercholesterolemia, cholesterol gallstone, hypertriglyceridemia, hypertension, cataract, renal disease, neurological disorder, chronic inflammatory disease, and infectious disease.

6. A treatment method of metabolic disease or complications thereof containing the step of administering a therapeutically effective dose of the pterocarpan compound represented by Formula 1 or the pharmaceutically acceptable salt thereof of claim 1 to a patient in need of treatment.

7. A health functional food composition for the prevention or improvement of metabolic disease or complications thereof containing the pterocarpan compound represented by Formula 1 or the pharmaceutically acceptable salt thereof of claim 1 as an active ingredient.

8. The health functional food composition for the prevention or improvement of metabolic disease or complications thereof according to claim 7, wherein the metabolic disease is selected from the group consisting of diabetes, hyperlipidemia, atherosclerosis, fatty liver, and obesity; and the complication is selected from the group consisting of coronary artery disease, angina pectoris, carotid artery disease, stroke, cerebral arteriosclerosis, hypercholesterolemia, cholesterol stone, hypertriglyceridemia, hypertension, cataract, renal disease, neurological disorder, chronic inflammatory disease, and infectious disease.

9. An anti-oxidative pharmaceutical composition containing the pterocarpan compound represented by Formula 1 or the pharmaceutically acceptable salt thereof of claim 1 as an active ingredient.

10. An anti-oxidative health functional food composition containing the pterocarpan compound represented by Formula 1 or the pharmaceutically acceptable salt thereof of claim 1 as an active ingredient.

11. An anti-oxidative cosmetic composition containing the pterocarpan compound represented by Formula 1 or the pharmaceutically acceptable salt thereof of claim 1 as an active ingredient.

12. A anti-oxidative feed additive containing the pterocarpan compound represented by Formula 1 or the pharmaceutically acceptable salt thereof of claim 1 as an active ingredient.
